SEQUENCE LISTING (1 sequence)

SEQ ID NO: 1 - DNA, 19 bp, Artificial Sequence
  RNA (1)...(4)
  RNA-DNA chimeric primer
ccttacaccgtttctcatt

(19) United States
(12) Patent Application Publication
(10) Pub. No.: US 2012/0053064 A1
(43) Pub. Date: Mar. 1, 2012

(54) DETERMINING THE IDENTITY OF TERMINAL NUCLEOTIDES

(76) Inventors: William E. Highsmith, JR., Rochester, MN (US); Laura K. Courteau, Dodge Center, MN (US); Jennifer L. Winters, Rochester, MN (US); Leonard M. Holtegaard, Rochester, MN (US)

(21) Appl. No.: 13/215,923

(22) Filed: Aug. 23, 2011

Related U.S. Application Data
(60) Provisional application No. 61/376,472, filed on Aug. 24, 2010.

(51) Int. Cl. C40B 20/08; C40B 50/06; C12P 19/34

(52) U.S. Cl. 506/6; 435/91.53; 506/26

(57) ABSTRACT

This document provides methods and materials for determining the identity of the terminal nucleotide (e.g., the 3′ terminal nucleotide) of a nucleic acid molecule. For example, methods and materials for using RNA-DNA chimeric primers during a primer extension reaction followed by treatment with one or more RNAse enzymes to prepare nucleic acid molecules that can be analyzed to identify the terminal nucleotides added to the RNA-DNA chimeric primers during the primer extension are provided.

### CROSS-REFERENCE TO RELATED APPLICATION

This application claims the benefit of U.S. Provisional Application No. 61/376,472, filed Aug. 24, 2010. The content of the foregoing application is hereby incorporated by reference in their entireties.

### BACKGROUND

1. Technical Field

This document relates to methods and materials involved using a primer extension technique to determine the identity of a specific nucleotide change in a nucleic acid sequence. Such changes can represent pathogenic mutations or single nucleotide polymorphisms (SNPs). The identity of the nucleotide under investigation can be determined by the identification of the terminal nucleotide (e.g., the 3′ terminal nucleotide) of a nucleic acid primer molecule. For example, this document relates to methods and materials involved in using RNA-DNA chimeric primers and one or more RNAse enzymes to increase the number of SNPs or mutations that can be identified simultaneously during, for example, a primer extension technique.

2. Background

Determining polymorphic bases at known single nucleotide polymorphism (SNP) sites and detecting a mutant base at a specific locus are common needs in molecular epidemiology and molecular diagnostics. A large number of methods have been developed to accomplish this, and many are commercially available. For example, ABI's PRISM® SNaPshot® ddNTP Primer Extension Kit and Sequenom's MassArray™ iPLEX® GOLD SNP Genotyping techniques are designed to determine the identity of nucleotides present at SNP sites.

### SUMMARY

This document provides methods and materials for determining the identity of the terminal nucleotide (e.g., the 3′ terminal nucleotide) of a nucleic acid molecule. The methods and materials provided herein can be used to increase the number of SNPs or mutations that can be identified simultaneously using a primer extension technique.

As described herein, this document provides methods and materials for using RNA-DNA chimeric primers during a primer extension reaction followed by treatment with one or more RNAse enzymes to prepare nucleic acid molecules that can be analyzed to identify the terminal nucleotides added to the RNA-DNA chimeric primers during the primer extension. RNA-DNA chimeric primers can be designed to be of sufficient length (e.g., greater than 15, 20, 25, 30, or more nucleotides) to provide sufficient sequence specificity for annealing to a desired nucleic acid template. Once the primer extension reaction is completed and the extended primers are treated with one or more RNAse enzymes, the remaining portion of the extended primer can be of sufficient length (e.g., between 4 and 25 nucleotides, between 6 and 25 nucleotides, between 8 and 25 nucleotides, between 10 and 25 nucleotides, between 5 and 20 nucleotides, between 5 and 15 nucleotides, between 8 and 20 nucleotides, or between 10 and 20 nucleotides) to be analyzed by matrix-assisted laser desorption/ionization (MALDI) time-of-flight (TOF) mass spectrometry or by capillary electrophoresis such that the identity of the terminal 3′ nucleotide can be determined. The use of longer RNA-DNA chimeric primers during the primer extension reaction and shorter portions of the extended primers during the process of determining the identity of the terminal nucleotide can allow a large number of different SNP sites (e.g., more than 10, 20, or different SNP sites) to be analyzed with a single assay (e.g., a multiplex primer extension reaction followed by, for example, a single MALDI-TOF mass spectrometry procedure). Having the ability to perform large multiplexed assays can allow clinicians, researchers, and other biomedical professionals to assess many SNP positions within a genome in a quick, accurate, and efficient manner.

In one aspect, this document features a method for preparing nucleic acid for determining the identity of a terminal nucleotide following primer extension. The method comprises, or consists essentially of, (a) contacting a nucleic acid template with a primer in the presence of a chain terminating nucleotide mixture and a DNA polymerase to form an extended primer, wherein the primer is capable of annealing to a region of the nucleic acid template, wherein the primer comprises a 5′ end region and a 3′ end region, wherein at least 40 percent of the nucleotides of the 5′ end region are ribonucleotides, wherein the nucleotides of the 3′ end region are deoxyribonucleotides, and wherein the extended primer comprises the primer and one chain terminating nucleotide of the mixture added to the 3′ end of the primer, and (b) contacting the extended primer of step (a) with an RNAse under conditions wherein the ribonucleotides of the extended primer are removed from the 3′ end region of the extended primer, thereby forming a nucleic acid comprising the 3′ end region of the primer and the one chain terminating nucleotide of the mixture added to the 3′ end of the primer. The nucleic acid template can be a nucleic acid amplification product. The nucleic acid template can be a nucleic acid molecule amplified from genomic nucleic acid. The nucleic acid template can be a nucleic acid molecule amplified from human genomic nucleic acid. The sequence of the primer can be complementary to the sequence of the region of the nucleic acid template. The chain terminating nucleotide mixture can comprise ddATPs, ddTTPs, ddGTPs, and ddCTPs. The DNA polymerase can be a thermostabile DNA polymerase. The primer can be between 18 and 50 nucleotides in length. The primer can be between 16 and 24 nucleotides in length. The 5′ end region of the primer can be between 4 and 17 nucleotides in length. The 5′ end region of the primer can be between 5 and 15 nucleotides in length. The 3′ end region of the primer can be between three and 40 nucleotides in length. The 3′ end region of the primer can be between 10 and 20 nucleotides in length. The 3′ end region of the primer can be between 11 and 15 nucleotides in length. The primer can consist of the 5′ end region and the 3′ end region. In some cases, all of the nucleotides of the 5′ end region can be ribonucleotides. At least every other nucleotide of the 5′ end region can be a ribonucleotide. The RNAse can be RNase A or RNase T1. The step (b) can comprise contacting the extended primer of step (a) with a mixture of RNAses. The maximum length of contiguous nucleotides of the 5′ end region of the extended primer remaining following the step (b) can be less than five nucleotides. The maximum length of contiguous nucleotides of the 5′ end region of the extended primer remaining following the step (b) can be less than four nucleotides. The maximum length of contiguous nucleotides of the 5′ end region of the extended primer remaining following the step (b) can be less than three nucleotides. In some cases, no contiguous nucleotides of the 5′ end region of the extended primer remain following the step (b).

In another aspect, this document features a method for preparing a collection of nucleic acid molecules for determining the identity of the terminal nucleotides following primer extension. The method comprises, or consists essentially of, (a) contacting one or more nucleic acid templates with a collection of primers in the presence of a chain terminating nucleotide mixture and a DNA polymerase to form a collection of extended primers, wherein each of the primers of the collection of primers is capable of annealing to a different region of the one or more nucleic acid templates, wherein each of the primers of the collection of primers comprises a 5′ end region and a 3′ end region, wherein at least 40 percent of the nucleotides of the 5′ end region are ribonucleotides, wherein the nucleotides of the 3′ end region are deoxyribonucleotides, and wherein each of the extended primers of the collection of extended primers comprises one of the primers of the collection of primers and one chain terminating nucleotide of the mixture added to the 3′ end of the one of the primers, and (b) contacting the collection of extended primers of step (a) with an RNAse under conditions wherein the ribonucleotides of each of the extended primers of the collection of extended primers are removed from the 3′ end region of each of the extended primers of the collection of extended primers, thereby forming a collection of nucleic acid molecules, wherein each of the nucleic acid molecules of the collection of nucleic acid molecules comprises the 3′ end region of one of the primers of the collection of primers and the one chain terminating nucleotide of the mixture added to the 3′ end of the one of the primers of the collection of primers. The length of each of the nucleic acid molecules of the collection of nucleic acid molecules can be different. Each of the one or more nucleic acid templates can be a nucleic acid amplification product. Each of the one or more nucleic acid templates can be a nucleic acid molecule amplified from genomic nucleic acid. Each of the one or more nucleic acid templates can be a nucleic acid molecule amplified from human genomic nucleic acid. The sequence of each of the primers of the collection of primers can be complementary to the sequence of a different region of the one or more nucleic acid templates. The chain terminating nucleotide mixture can comprise ddATPs, ddTTPs, ddGTPs, and ddCTPs. The DNA polymerase can be a thermostabile polymerase. Each of the primers of the collection of primers can be between 18 and 50 nucleotides in length. Each of the primers of the collection of primers can be between 16 and 24 nucleotides in length. The 5′ end region can be between 4 and 17 nucleotides in length. The 5′ end region can be between 5 and 15 nucleotides in length. The 3′ end region can be between three and 40 nucleotides in length. The 3′ end region can be between 10 and 20 nucleotides in length. The 3′ end region can be between 11 and 15 nucleotides in length. Each of the primers of the collection of primers can consist of the 5′ end region and the 3′ end region. In some cases, all of the nucleotides of the 5′ end region can be ribonucleotides. At least every other nucleotide of the 5′ end region can be a ribonucleotide. The RNAse can be RNase A or RNase T1. The step (b) can comprise contacting the collection of extended primers of step (a) with a mixture of RNAses. The maximum length of contiguous nucleotides of the 5′ end region of each of the extended primers of the collection of extended primers remaining following the step (b) can be less than five nucleotides. The maximum length of contiguous nucleotides of the 5′ end region of each of the extended primers of the collection of extended primers remaining following the step (b) can be less than four nucleotides. The maximum length of contiguous nucleotides of the 5′ end region of each of the extended primers of the collection of extended primers remaining following the step (b) can be less than three nucleotides. In some cases, no contiguous nucleotides of the 5′ end region of each of the extended primers of the collection of extended primers remain following the step (b). The chain terminating nucleotides of the mixture can be fluorescently labeled dideoxynucleotides, where each dideoxynucleotide is labeled with a different fluorophore at either the nucleotide base or the ribose sugar moiety. Each of the ddATPs, the ddTTPs, the ddGTPs, and the ddCTPs can be labeled with a different fluorescent label.

In another aspect, this document features a method for determining the identity of nucleotide at a position along a nucleic acid. The method comprises, or consist essentially of, (a) contacting a nucleic acid template with a primer in the presence of a chain terminating nucleotide mixture and a DNA polymerase to form an extended primer, wherein the primer is capable of annealing to a region of the nucleic acid template that is 5′ of the position such that the first nucleotide in the 3′ direction of the primer along the nucleic acid template is at the position, wherein the primer comprises a 5′ end region and a 3′ end region, wherein at least 40 percent of the nucleotides of the 5′ end region are ribonucleotides, wherein the nucleotides of the 3′ end region are deoxyribonucleotides, and wherein the extended primer comprises the primer and one chain terminating nucleotide of the mixture added to the 3′ end of the primer, (b) contacting the extended primer of step (a) with an RNAse under conditions wherein the ribonucleotides of the extended primer are removed from the 3′ end region of the extended primer, thereby forming a nucleic acid comprising the 3′ end region of the primer and the one chain terminating nucleotide of the mixture added to the 3′ end of the primer, and (c) determining the identity of the 3′ terminal nucleotide of the nucleic acid formed in the step (b). The step (c) can be performed using MALDI-TOF mass spectrometry. The chain terminating nucleotide mixture can comprise ddATPs, ddTTPs, ddGTPs, and ddCTPs, each of which comprises a different fluorescent label, and wherein the identity of the 3′ terminal nucleotide of the nucleic acid formed in the step (b) is determined based on the type of fluorescent label of a dideoxynucleotide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a mass spectrometry trace (Sequenom Mass Array trace) of an RNA-DNA chimeric primer not treated with RNAse enzymes.

FIG. 2 is a mass spectrometry trace (Sequenom Mass Array trace) of an RNA-DNA chimeric primer after treatment with a mixture of RNAse enzymes.

### DETAILED DESCRIPTION

This document provides methods and materials for determining the identity of the terminal nucleotide (e.g., the 3′ terminal nucleotide) of a nucleic acid molecule. For example, this document provides methods and materials for using RNA-DNA chimeric primers during a primer extension reaction followed by treatment with one or more RNAse enzymes to prepare nucleic acid molecules that can be analyzed to identify the terminal nucleotides added to the RNA-DNA chimeric primers during the primer extension.

As described herein, an RNA-DNA chimeric primer can be designed to be of sufficient length to provide sufficient sequence specificity for annealing to a desired position along a nucleic acid template. For example, the length of an RNA-DNA chimeric primer can be between 15 and 75 nucleotides (e.g., between 20 and 50 nucleotides, between 20 and 40 nucleotides, between 20 and 30 nucleotides, between 25 and 50 nucleotides, between 30 and 50 nucleotides, between 15 and 40 nucleotides, or between 20 and 75 nucleotides). In general, the sequence of an RNA-DNA chimeric primer provided herein is designed such that the terminal 3′ nucleotide of the primer is immediately adjacent to a SNP site or mutation site to be analyzed so that extension of the primer by one nucleotide results in the addition of the one nucleotide that is complementary to the nucleotide present in the nucleic acid template at that SNP site or mutation site. The use of such an RNA-DNA chimeric primer during a primer extension reaction that uses a mixture of chain terminating nucleotides (e.g., dideoxynucleotides and/or acyclo-nucleotides), as opposed to a mixture of deoxynucleotides, results in only one nucleotide being added to the 3′ end of the RNA-DNA chimeric primer to form an extended primer. Since the RNA-DNA chimeric primer is designed such that the added terminal 3′ nucleotide corresponds to which ever nucleotide is present at the SNP site or mutation site of the nucleic acid template, then the identity of the nucleotide at the SNP site or mutation site can be determined by determining the identity of the added terminal 3′ nucleotide of the extended primer.

An RNA-DNA chimeric primer provided herein can include a 5′ end region and a 3′ end region. The 5′ end region includes the 5′ terminal nucleotide and can extend toward the 3′ terminal nucleotide any distance provided that the primer has at least three, four, or five nucleotides of the 3′ end region. The 3′ end region includes the 3′ terminal nucleotide and can extend toward the 5′ terminal nucleotide any distance provided that the primer has at least one, two, three, four, or five nucleotides of the 5′ end region. For example, an RNA-DNA chimeric primer provided herein can have a length of 40 nucleotides with the first 30 nucleotides being the 5′ end region and the last 10 nucleotides being the 3′ end region.

As described herein, at least 40 (e.g., at least 45, 50, 55, 60, 65, 70, 75, 80, 85, 90, 95, or more) percent of the nucleotides of the 5′ end region of an RNA-DNA chimeric primer provided herein can be ribonucleotides. In some cases, every nucleotide of the 5′ end region of an RNA-DNA chimeric primer provided herein can be a ribonucleotide. In some cases, at least every other nucleotide of the 5′ end region of an RNA-DNA chimeric primer provided herein can be ribonucleotides (e.g., one out of every two nucleotides, two out of every three nucleotides, three out of every four nucleotides, or four out of every five nucleotides). The nucleotides of the 3′ end region of an RNA-DNA chimeric primer provided herein can be deoxyribonucleotides. In some cases, none of the nucleotides of the 3′ end region of an RNA-DNA chimeric primer provided herein is a ribonucleotide. In some cases, every nucleotide of the 5′ end region of an RNA-DNA chimeric primer provided herein can be a ribonucleotide, and every nucleotide of the 3′ end region of the RNA-DNA chimeric primer can be a deoxyribonucleotide.

The 5′ end region and the 3′ end region of an RNA-DNA chimeric primer provided herein can be any appropriate length. For example, the 5′ end region of an RNA-DNA chimeric primer provided herein can be between four and 25 nucleotides (e.g., between four and 23 nucleotides, between four and 21 nucleotides, between four and 20 nucleotides, between five and 25 nucleotides, between six and 25 nucleotides, or between seven and 25 nucleotides). The 3′ end region of an RNA-DNA chimeric primer provided herein can be between three and 30 or more nucleotides (e.g., between four and 30 nucleotides, between five and 30 nucleotides, between six and 30 nucleotides, between seven and 30 nucleotides, between eight and 30 nucleotides, between nine and 30 nucleotides, between ten and 30 nucleotides, between five and 50 nucleotides, or between five and 60 nucleotides).

An RNA-DNA chimeric primer provided herein can be used to perform a primer extension reaction that involves using a mixture of chain terminating nucleotides (e.g., dideoxynucleotides and/or acyclo-nucleotides), as opposed to a mixture of deoxynucleotides, so that only one nucleotide is added to the RNA-DNA chimeric primer to form an extended primer. In some cases, an appropriate primer extension reaction can be performed using one or more RNA-DNA chimeric primers provided herein and chain terminating nucleotides (e.g., dideoxynucleotides and/or acyclo-nucleotides), not deoxynucleotides. In some cases, the deoxynucleotides used in the primer extension reaction can be fluorescently labeled such that each of the four different chain terminating nucleotides (e.g., dideoxynucleotides or acyclo-nucleotides) have a different fluorescent label. Examples of DNA polymerases (e.g., themostabile DNA polymerases with high fidelity) that can be used to perform a primer extension reaction include, without limitation, Thermo Sequenase™ (GE Healthcare), Therminator, Therminator II, and Therminator III (NEB BioLabs).

Once the primer extension reaction is completed, the resulting extended primers can be treated with one or more RNAse enzymes such that the ribonucleotides of the extended primer are removed from the 3′ end region that lacks ribonucleotides.

In some cases, a mixture of two, three, four, five, or more difference RNAse enzymes can be used to remove ribonucleotides (and any other type of nucleotides that are 5′ of a ribonucleotide) from the 3′ end region of the extended primer that lacks ribonucleotides. Examples of RNAse enzymes that can be used as described herein include, without limitation, RNase Cocktail (Ambion), RNAse1 (Ambion), RNAse H (Ambion), RNase I, RNase III, RNase III, RNase P, RNase PhyM, RNase T1, RNase T2, RNase U2, RNase V1, RNase V, PNPase, RNase PH, RNase II, RNase R, RNase D, RNase T, Oligoribonuclease, Exoribonuclease I, and Exoribonuclease II. Any appropriate RNAse enzyme reaction can be performed. For example, about 1.0 μL of RNase Cocktail (containing RNase A 500 U/mL, RNase T1 at 20,000 U/mL) can be added to about 5.0 μL of extended chimeric product and incubated at about 37° C. for about one hour.

Once the extended primers are treated with one or more RNAse enzymes, the remaining 3′ portion of the extended primer can be analyzed to determine the identity of the 3′ terminal nucleotide that was added during the primer extension reaction. When fluorescently labeled chain terminating nucleotides (e.g., dideoxynucleotides or acyclo-nucleotides) are used in the primer extension reaction, the identity of the 3′ terminal nucleotide that was added during the primer extension reaction can be determined based on the type of fluorescent label. In such cases, methods such as capillary electrophoresis with laser induced fluorescence detection can be used to identify the 3′ terminal nucleotide. When labeled or unlabeled chain terminating nucleotides (e.g., dideoxynucleotides or acyclo-nucleotides) are used in the primer extension reaction, the identity of the 3′ terminal nucleotide that was added during the primer extension reaction can be determined based on molecular weight. In such cases, methods such as MALDI-TOF mass spectrometry can be used to identify the 3′ terminal nucleotide. Any appropriate MALDI-TOF mass spectrometry can be used including, without limitation, SELDI (Ciphergen). For example, Sequenom Mass Array™ can be used. In this method, a single mass-tagged terminating nucleotide (e.g., dideoxy and/or acyclo) complementary to the base at, e.g., the polymorphic position in the DNA template can be added to the 3′ end of an olgonucleotide primer by polymerase extension. In this case, the mass tagged terminators can include: acyC (247 daltons), acyT (262 daltons), ddA (297 daltons), and ddG (313 daltons). Extension reaction products can be treated to remove Na, Mg, and K adducts. An ammoniated clean resin (Bio-Rad, Hercules, Calif.) treatment can be used to remove Na, Mg, and K adducts. Then, a portion of the sample can be dispensed to a matrix pad of a SpectroCHIP II (Sequenom, Inc) using a RS-1000 Nanodispenser (Sequenom, Inc.). Using the Sequenom Mass Array™, MALDI-TOP can be performed on the samples in which the laser desorption occurs with a nitrogen laser, and positive ions of the nucleic acid in linear mode can be detected. Raw data from the mass spectrometer can be imported by the SpectraAQUIRE software (Sequenom, Inc.) for each sample pad and analyzed by the MassARRAY System Typer version 4.0 software (Sequenom Inc.). Visual inspection of the mass of the extended primer in the Spectra can be performed to identify the added nucleotide, and thus identify the identity of the base at the polymorphic position in the template.

As described herein, many different RNA-DNA chimeric primers provided herein can be designed and used in parallel in a single assay. For example, 10, 20, 30, or more different RNA-DNA chimeric primers provided herein can be designed such that each can identify the nucleotide present at a different SNP site or mutation site. Such a collection of RNA-DNA chimeric primers can be used together in a single primer extension reaction, a single RNAse reaction, and/or a single interrogation procedure (e.g., a single MALDI-TOF mass spectrometry run). When using many different RNA-DNA chimeric primers to assess many different SNP sites and/or mutation sites at the same time, the 3′ end regions of each different RNA-DNA chimeric primer can be designed such that the overall molecular weight of each different extended primer (following RNAse treatment) does not overlap, thereby allowing each different primer to be located in a mass spectrometry trace.

The invention will be further described in the following examples, which do not limit the scope of the invention described in the claims.

### EXAMPLES

### Example 1

### Treatment of RNA-DNA Chimeric Primers with RNAses Results in Removal of the Ribonucleotides

An RNA-DNA chimeric primer was designed and prepared to have the following sequence: CCTTACACCGTTTCTCATT (SEQ ID NO:1). The underlined nucleotides of this RNA-DNA chimeric primer (first four nucleotides) were ribonucleotides, while the remaining nucleotides were deoxyribonucleotides. It is understood that RNA uses uracil (U) in place of thymine (T) even though thymine is indicated in some instances herein for a ribonucleotide. This RNA-DNA chimeric primer was designed for the detection of the 2143delT mutation in the CFTR gene. A Sequenom Mass Array analysis was performed using the RNA-DNA chimeric primer without treatment with RNAse enzymes and the RNA-DNA chimeric primer following treatment with RNAse enzymes. The RNAse enzyme treatment was performed using an RNase Cocktail (Ambion) and the following conditions. 1.0 μL of RNase Cocktail (containing RNase A 500 U/mL, RNase T1 at 20,000 U/mL) was added to 7.0 μL of extended chimeric product. This reaction was incubated at 37° C. for 1 hour. The sample was then processed as usual.

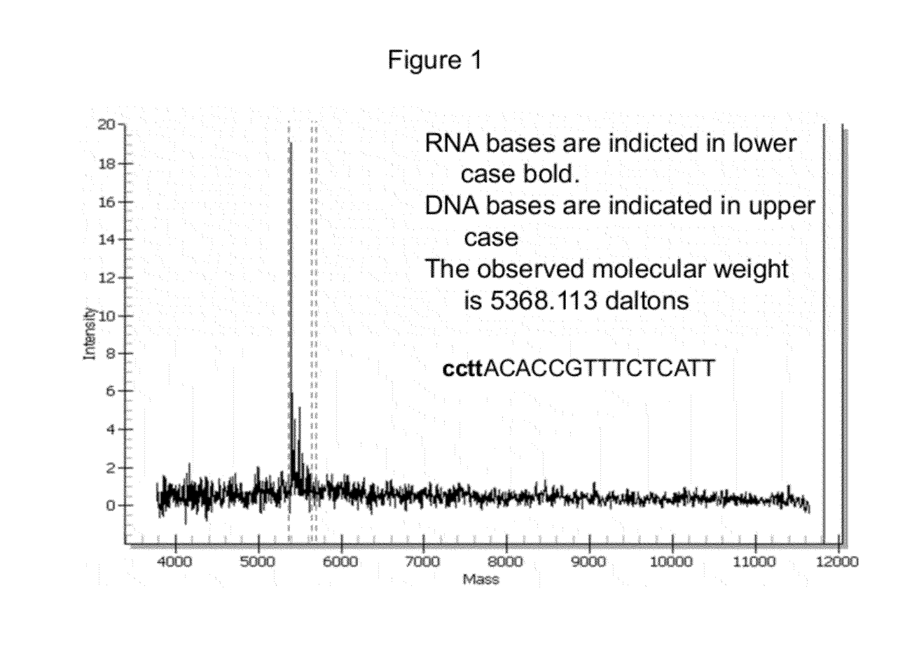

The Sequenom Mass Array trace for the RNA-DNA chimeric primer not treated with RNAse enzymes resulted in a peak corresponding to the molecular weight of the RNA-DNA chimeric primer (FIG. 1). The dotted lines of FIG. 1 were included to represent the expected masses for the unextended DNA only primer and the expected extended DNA only primers resulting from wild-type or mutant alleles at the 2143delT location. The mass of the RNA-DNA chimeric primer is slightly higher than that expected for the unextended DNA only primer due to the presence of an additional —OH group on each of the four ribonucleotides.

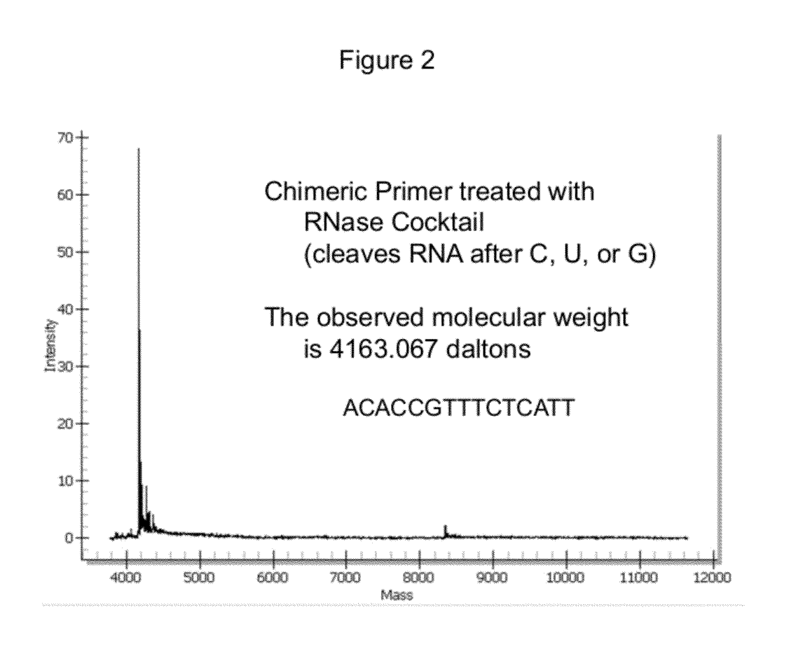

The Sequenom Mass Array trace for the RNA-DNA chimeric primer following treatment with RNAse enzymes resulted in a peak corresponding to the mass of a nucleic acid molecule created by the removal of the first four ribonucleotides of SEQ ID NO:1 (FIG. 2). These results demonstrate that the RNAse reaction was complete in that no residue material at the original molecular weight of the RNA-DNA chimeric primer was observed. These results also demonstrate that the RNAse reaction was clean in that no peaks were observed between the original molecular weight of the RNA-DNA chimeric primer and the molecular weight of the DNA portion of the RNA-DNA chimeric primer.

Taken together, these results demonstrate that RNA-DNA chimeric primers can be used to identify the nucleotides present at SNP sites or mutation sites in a manner that provides increased sequence specificity during the primer extension reaction (via longer primers) and provides the ability to assess more SNP sites or mutation sites in a single mass spectrometry assay (via shorter primers following RNAse treatment).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for preparing nucleic acid for determining the identity of a terminal nucleotide following primer extension, wherein said method comprises:
(a) contacting a nucleic acid template with a primer in the presence of a chain terminating nucleotide mixture and a DNA polymerase to form an extended primer, wherein said primer is capable of annealing to a region of said nucleic acid template, wherein said primer comprises a 5′ end region and a 3′ end region, wherein at least 40 percent of the nucleotides of said 5′ end region are ribonucleotides, wherein the nucleotides of said 3′ end region are deoxyribonucleotides, and wherein said extended primer comprises said primer and one chain terminating nucleotide of said mixture added to the 3′ end of said primer, and
(b) contacting said extended primer of step (a) with an RNAse under conditions wherein the ribonucleotides of said extended primer are removed from the 3′ end region of said extended primer, thereby forming a nucleic acid comprising said 3′ end region of said primer and said one chain terminating nucleotide of said mixture added to the 3′ end of said primer.

2. The method of claim 1, wherein said nucleic acid template is a nucleic acid amplification product.

3. The method of claim 1, wherein said chain terminating nucleotide mixture comprises ddATPs, ddTTPs, ddGTPs, and ddCTPs.

4. The method of claim 1, wherein said primer is between 18 and 50 nucleotides in length.

5. The method of claim 1, wherein said 5′ end region of said primer is between 4 and 17 nucleotides in length.

6. The method of claim 1, wherein said 3′ end region of said primer is between three and 40 nucleotides in length.

7. The method of claim 1, wherein at least every other nucleotide of said 5′ end region is a ribonucleotide.

8. The method of claim 1, wherein the maximum length of contiguous nucleotides of said 5′ end region of said extended primer remaining following said step (b) is less than five nucleotides.

9. A method for preparing a collection of nucleic acid molecules for determining the identity of the terminal nucleotides following primer extension, wherein said method comprises:
(a) contacting one or more nucleic acid templates with a collection of primers in the presence of a chain terminating nucleotide mixture and a DNA polymerase to form a collection of extended primers, wherein each of the primers of said collection of primers is capable of annealing to a different region of said one or more nucleic acid templates, wherein each of the primers of said collection of primers comprises a 5′ end region and a 3′ end region, wherein at least 40 percent of the nucleotides of said 5′ end region are ribonucleotides, wherein the nucleotides of said 3′ end region are deoxyribonucleotides, and wherein each of the extended primers of said collection of extended primers comprises one of the primers of said collection of primers and one chain terminating nucleotide of said mixture added to the 3′ end of said one of the primers, and
(b) contacting said collection of extended primers of step (a) with an RNAse under conditions wherein the ribonucleotides of each of the extended primers of said collection of extended primers are removed from the 3′ end region of each of the extended primers of said collection of extended primers, thereby forming a collection of nucleic acid molecules, wherein each of the nucleic acid molecules of said collection of nucleic acid molecules comprises said 3′ end region of one of the primers of said collection of primers and said one chain terminating nucleotide of said mixture added to the 3′ end of said one of the primers of said collection of primers.

10. The method of claim 9, wherein the length of each of said nucleic acid molecules of said collection of nucleic acid molecules is different.

11. The method of claim 9, wherein said chain terminating nucleotide mixture comprises ddATPs, ddTTPs, ddGTPs, and ddCTPs.

12. The method of claim 9, wherein each of the primers of said collection of primers is between 18 and 50 nucleotides in length.

13. The method of claim 9, wherein said 5′ end region is between 4 and 17 nucleotides in length.

14. The method of claim 9, wherein said 3′ end region is between three and 40 nucleotides in length.

15. The method of claim 9, wherein at least every other nucleotide of said 5′ end region is a ribonucleotide.

16. The method of claim 9, wherein the maximum length of contiguous nucleotides of said 5′ end region of each of the extended primers of said collection of extended primers remaining following said step (b) is less than five nucleotides.

17. The method of claim 9, wherein the chain terminating nucleotides of said mixture are fluorescently labeled dideoxynucleotides, where each type of dideoxynucleotide of said mixture is labeled with a different fluorophore at either the nucleotide base or the ribose sugar moiety.

18. A method for determining the identity of nucleotide at a position along a nucleic acid, wherein said method comprises:
(a) contacting a nucleic acid template with a primer in the presence of a chain terminating nucleotide mixture and a DNA polymerase to form an extended primer, wherein said primer is capable of annealing to a region of said nucleic acid template that is 5′ of said position such that the first nucleotide in the 3′ direction of said primer along said nucleic acid template is at said position, wherein said primer comprises a 5′ end region and a 3′ end region, wherein at least 40 percent of the nucleotides of said 5′ end region are ribonucleotides, wherein the nucleotides of said 3′ end region are deoxyribonucleotides, and wherein said extended primer comprises said primer and one chain terminating nucleotide of said mixture added to the 3′ end of said primer,
(b) contacting said extended primer of step (a) with an RNAse under conditions wherein the ribonucleotides of said extended primer are removed from the 3′ end region of said extended primer, thereby forming a nucleic acid comprising said 3′ end region of said primer and said one chain terminating nucleotide of said mixture added to the 3′ end of said primer, and (c) determining the identity of the 3′ terminal nucleotide of said nucleic acid formed in said step (b).

19. The method of claim 18, wherein said step (c) is performed using MALDI-TOF mass spectrometry.

20. The method of claim 18, wherein said chain terminating nucleotide mixture comprises ddATPs, ddTTPs, ddGTPs, and ddCTPs, each of which comprises a different fluorescent label, and wherein the identity of said 3′ terminal nucleotide of said nucleic acid formed in said step (b) is determined based on the type of fluorescent label of a dideoxynucleotide.
